(19) United States
(12) Patent Application Publication
(10) Pub. No.: US 2005/0165068 A1
(43) Pub. Date: Jul. 28, 2005

(54) Compounds, in particularly of urea derivatives or esters of haloacetamidobenzoic acid and use thereof for the treatment of parasitic diseases

(76) Inventor: Patrice Lepape, Vertou (FR)

Correspondence Address: YOUNG & THOMPSON, 745 SOUTH 23RD STREET, 2ND FLOOR, ARLINGTON, VA, 22202, US

(21) Appl. No.: 10/503,095

(22) PCT Filed: Jan. 31, 2003
(86) PCT No.: PCT/FR03/00289

(30) Foreign Application Priority Data
Jan. 31, 2002 (FR) 0201151

(51) Int. Cl. A61K 31/44; A61K 31/277; A61K 31/165

(52) U.S. Cl. 514/352; 514/616; 514/520

(57) ABSTRACT

The invention relates to the use of a compound of general formula (I) for the production of a medicament used for treating parasitic diseases, particularly leishmaniasis.

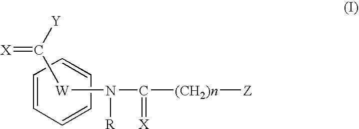

The present invention relates to the use of compounds, in particular derivatives of haloacetamidobenzoic acid for the treatment of parasitic maladies, in particular protozooses and more particularly for the treatment of leishmaniosis, paludism and tripanosomiases.

Paludism is an erythropathy due to Plasmodium transmitted by mosquito. More than 2 million subjects are exposed to a risk of transmission. According to the OMS, there would be 300 millions of new cases per year and it is estimated that several millions will die per year in central Africa alone. Every 9 seconds, someone dies of paludism. One of the species, Plasmodium falciparum, is responsible for neuropaludism, mortal encephalopathy in the absence of treatment. The appearance of resistance to chloroquinine in wide geographic regions has frustrated the prophylaxis and the treatment of this infection.

Chagas disease is an anthropozoonosis due to Trypanosoma cruzi transmitted by blood sucking insects, bedbugs. It occurs in Latin America, where it affects 20 million individuals. This malady is manifested by meningo-encephalitis sometimes deadly, as well as cardiopathies and chronic encephalopathies. The number of deaths is estimated at 50,000 per year. The antiparasitic treatment. relies on nifurtimox (LAMPIT®) with quite variable activity on the acute phase of the malady and inactive on the chronic phase.

Traditionally, leishmanioses are described as protozooses of the reticulo-endothelial system of mammals, such as humans, due to the Leishmania genus. The malady occurs over almost all of the world (88 countries) constituting a major problem of public health. In the endemic regions, the prevalence is estimated at more than 12 millions of cases and more than 400,000 new cases appear each year.

At present, 18 species of pathogens are known for humans. Leishmania donovani and L. infantum are responsible for visceral leishmaniosis, a potentially fatal clinical form, in the Mediterranean basin, the Maghreb, East Africa, India and South America. In 1993, the last epidemic in the Sudan was marked by 40,000 deaths. The agents of cutaneous leishmaniosis are L. major, L. tropica and L. aetiopica in the old world and L. mexicana, L. Panamensis and L. braziliensis in the new world. These protozoa generally transmitted by insect bites enter the cells such as the macrophages and the monocytes and give rise to bursting of the cells. The cutaneous form gives rise to ulcers whilst the visceral form gives rise to fever, weight loss and splenomegalia.

The treatment of leishmanioses relies essentially on the use of antimony derivatives which require the use of large doses, a parenteral use even in the exclusively cutaneous forms and a treatment of long duration. These derivatives are characterized also by renal and cardiac toxicity. Moreover, the susceptibility of strains of Leishmania to antimony is very variable and the cases of resistance are more and more numerous.

All of these considerations show the necessity and urgency of developing molecules which offer new therapeutic alternatives using an original mechanism of action.

Tubuline, the principal protein of microtubules, represents a cellular target for numerous drugs whose clinical effectiveness is established both in the fight against cancer and what is induced by numerous infectious agents such as parasites. The benzimidazole derivatives, known for their antimicrobial activity, are active against other protozoans such as Trichomonas vaginalis and Giardia. duodenalis.

Taxol, another antimicrotubular drug known at present for its anti-cancer activity, blocks the replication of Trypanosoma cruzi and induces a stop in the G2M phase of the cellular cycle of Leishmania donovani in the promastigotic phase. Although effective against Leishmania, taxol has not caught the attention of therapists because, if this type of drug is capable of altering microtubules of the parasite, it is also capable of altering to various degrees the microtubules of the cellular host.

Moreover, there are known, from U.S. Pat. No. 6,294,695, meta and para derivatives of haloacetamidobenzoic acid for application as inhibitors of the growth of tumoral cells. Nothing in this document suggests the use or of such compounds for applications to the treatment of parasitic maladies. Moreover, Table 1 column 18 of this document shows inactive or slightly active compounds for application to cancer therapy. However, these compounds are nevertheless interesting in the case of application to the treatment of parasitic maladies as will be described hereafter, in particular for a molecule called MF 29.

There are moreover known, from WO-A-0116357, compounds whose anti-parasitic activity is due to the inhibition of the enzymatic process. Such compounds have both a structure and a mechanism of action that is different from those of the compounds described in the present invention as shown in particular by the compound 105553 of FIG. 17A in which a cyclic group replaces the (CH₂)ₙ group of the present invention.

There are also known from EP-A-1 008 346, compounds derived from benzoquinone inhibitors of NF-KB and TNFα. Such compounds thus act as anti-inflammatory agents in particular by intervening in the production of cytokines by the lymphocytes T. Such compounds are thus adapted to treat inflammatory reactions associated with parasitic maladies but do not have any effect on the parasite itself.

International application WO 98/39323 discloses itself the use of thiophene derivatives as inhibitor agents for the polymerization of tubuline. Such compounds are however unable to distinguish the tubuline of the parasite from that of the cellular host.

US-2002/0065229, published later than the filing date of the present patent application, discloses only hypothetically the use of halogenated acetamidobenzoyl-ethyl-acetate derivatives during the treatment of parasitic maladies, whilst no test on a parasitic agent is recited in this document.

In conclusion, none of the mentioned documents discloses the use of anti-microtubular compounds of low toxicity, high activity, adapted to alter the microtubules of parasites without altering the microtubules of mammals.

An object of the present invention is to provide a new use of compounds for the treatment of parasitic maladies, in particular leishmanioses, of paludism and of tripanosomiases, no matter what their clinical form, these compounds being less toxic compared to known medications and being adapted to be used by oral administration not requiring hospitalization.

Another object of the present invention is to provide a new use of compounds for the treatment of parasitic maladies, in particular leishmanioses, paludism and tripanosomiases, these compounds causing alterations of morphology of the microtubules detected by sweeping electron microscopy, these alterations being accompanied by modifications of the organization of certain classes of microtubules and by a specificity of action on the microtobules depending on the microtubular composition of the cell that they affect.

Another object of the present invention is to provide a new use of compounds for the treatment of parasitic maladies, in particular leishmanioses, paludism and tripanosomiases, which interrupt the polymerization of the tubuline of the parasite without disturbing that of the host cell.

To this end, the invention has for its object the use of a compound of the general formula (I): 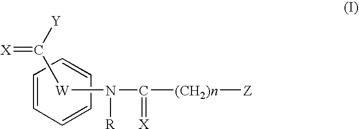
in which:
X represents an oxygen, sulfur or nitrogen atom,
R represents a hydrogen atom, a lower alkyl group,
n is 0, 1, 2, 3, 4 or 5,
Z represents a hydrogen atom, a halogen atom, lower trifluoroalkyl, trifluoromethoxyl, cyano, hydroxyl, alkoxyl, alkoxycarbonyl, carboxamido, phenyl, substituted phenyl, halophenyl,
Y represents a hydrogen atom, hydroxyl, alkoxyl, alkoxycarbonyl, carboxamido, phenyl, substituted phenyl, alkyl phenyl, halophenyl, a urea group or urea derivative, —N—R₁ group in which R₁ is an alkyl or heterocyclic group,
the symbol 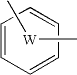
represents
either the phenyl nucleus
or the pyridine nucleus, the nitrogen being located in one of the 4 unsubstituted positions,
its enantiomers, its diastereoisomers and its pharmaceutically acceptable acid addition salts for the production of an anti-microtubular active agent for the treatment of parasitic maladies by inhibiting at least partially the polymerization of the tubuline of the parasite without inhibiting that of the host cell.

The invention also has for its object the use of a compound of general formula (I) of the mentioned type, its enantiomers, its diastereoisomers and its pharmaceutically acceptable acid addition salts for the production of a medication including at least one anti-microtubular active agent for the treatment of protozooses of the endothelial reticulo system of mammals.

The invention also has for its object the use of a compound of general formula (I) of the mentioned type, its enantiomers, its diastereoisomers and its pharmaceutically acceptable acid addition salts for the production of an anti-microtubular active agent for the treatment of leishmanioses, in particular human leishmaniosis, paludism and tripanosomiases.

It is to be noted that in the preceding, among the pharmaceutically acceptable acids, can be cited, by way of non-limiting example, hydrochloric acid, sulfuric acid, tartaric acid, maleic acid, fumeric acid, oxalic acid, methane-sulfonic acid, camphoric acid, ethane-sulfonic acid, etc . . .

The terms alkyl and alkoxyl designated linear or branched groups comprising 1 to 9 carbon atoms, preferably 1 to 6 carbon atoms.

The present invention also has for its object the use of a compound of the general formula (II) 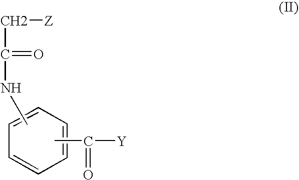
in which:

Z represents a halogen atom selected from the group consisting of chlorine, bromine, iodine and fluorine,

Y is a substituent selected from the group consisting of ethoxy, —NH—CO—NH—R in which R is hydrogen, an alkyl group of an aryl group, and by —N—R₁ in which R₁ is an alkyl or heterocyclic group, or its addition salts for the production of a medication for the treatment of parasitic maladies, in particular protozooses, more particularly leishmanioses, paludism or tripanosomiases.

The formula II above covers three types of compounds, namely: ortho compounds of the formula: 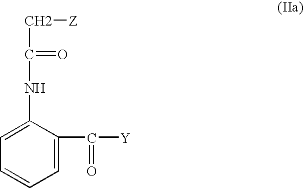
meta compounds of the formula: 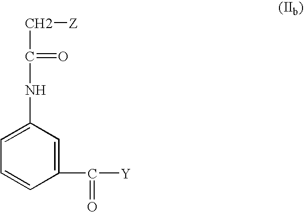
and para compounds of the formula: 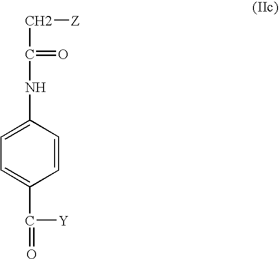
as well as their addition salts. In the formulas IIa, IIb, IIc above, Z and Y have the same definitions as those given for compounds of formula II.

The above compounds of formula II can also be used for the production of an anti-microtubular agent inhibiting at least partially the polymerization of the tubuline of the parasite without inhibiting that of the host cell.

The present invention also has for its object the use of a compound of the mentioned type of the general formula (I) or (II), in which this compound is associated with at least one excipient or pharmaceutically acceptable excipient or non-toxic inert vehicle permitting its administration by the oral route.

This compound can be present in the form of a solution or an aqueous suspension or in the form of dry tablets clad or not, gelatin coated pills, capsules, powders.

The invention also has for its object the use of a compound of general formula (I) or (II) of the mentioned type, in which the compound is associated with at least one pharmaceutically acceptable non-toxic excipient or inert vehicle permitting its administration topically in the form of cutaneous, transcutaneous or percutaneous applications. The compound can thus be present in the form of a pommade, a cream, a skin gel.

The invention also has for its object the use of a compound of general formula (I) or (II) of the mentioned type, in which the compound is associated with at least one pharmaceutically acceptable non-toxic excipient or inert vehicle permitting its administration parenterally, nasally or broncholly.

Generally, the content of the compound is selected for administration between 1 mg and 5 g/24 hours, preferably between 1 mg and 500 mg/24 hours.

The invention also has for its object a compound of general formula (I) 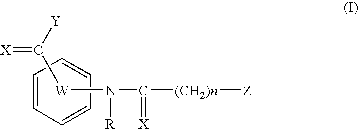
in which:
X represents oxygen, sulfur or nitrogen,
R represents hydrogen, lower alkyl,
n equals 0, 1, 2, 3, 4 or 5,
Z represents hydrogen, halogen, lower trifluoroalkyl, trifluoromethoxyl, cyano, hydroxyl, alkoxyl, alkoxycarbonyl, carboxamido, phenyl, substituted phenyl, halophenyl,
Y represents hydrogen, hydroxyl, alkoxyl, alkoxycarbonyl, carboxamido, phenyl, substituted phenyl, phenylalkyl, halophenyl, urea or urea derivative, —N—R₁ in which R₁ is alkyl or heterocyclic,
the sympbol 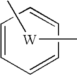
represents
either the phenyl nucleus
or the pyridine nucleus, the nitrogen being located at one of the 4 non-substituted positions,
its enantiomers, its diastereoisomers and its pharmaceutically acceptable acid addition salts,
excepting compounds in which R is hydrogne, X is oxygen, n=1, Z is halogen and Y is a substituent selected from the group consisting of ethoxy, —NH—CO—NH—R in which R is hydrogen, alkyl or aryl, and —N—R₁ in which R₁ is alkyl or heterocyclic or an addition salt thereof.

The invention also has for its object a medication comprising a physiologically acceptable support and a compound of formula (I) of the above type or an addition salt of the latter, in a quantity effective to treat parasitic maladies, in particular protozooses, preferably leishmanioses, paludism and tripanosomiases.

The invention will be better understood from a reading of the following description of examples of embodiment, with reference to the accompanying drawings, in which:

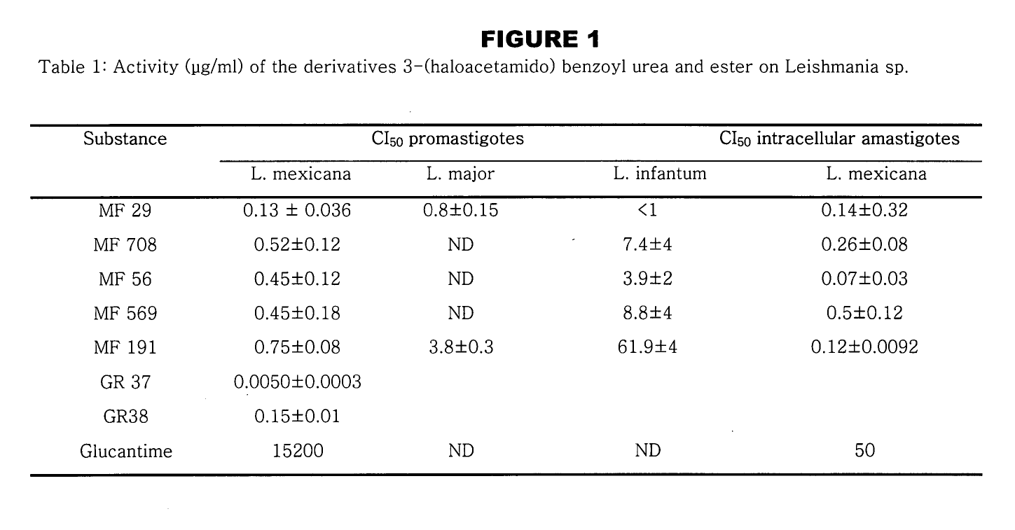

FIG. 1 represents, in tabular form, the in vitro anti-Leishmania activity of the molecules MF 29, MF 708, MF 56, MF 569, MF 191, GR 37, GR 38 and Glucantime™, said activities being expressed in μg/ml;

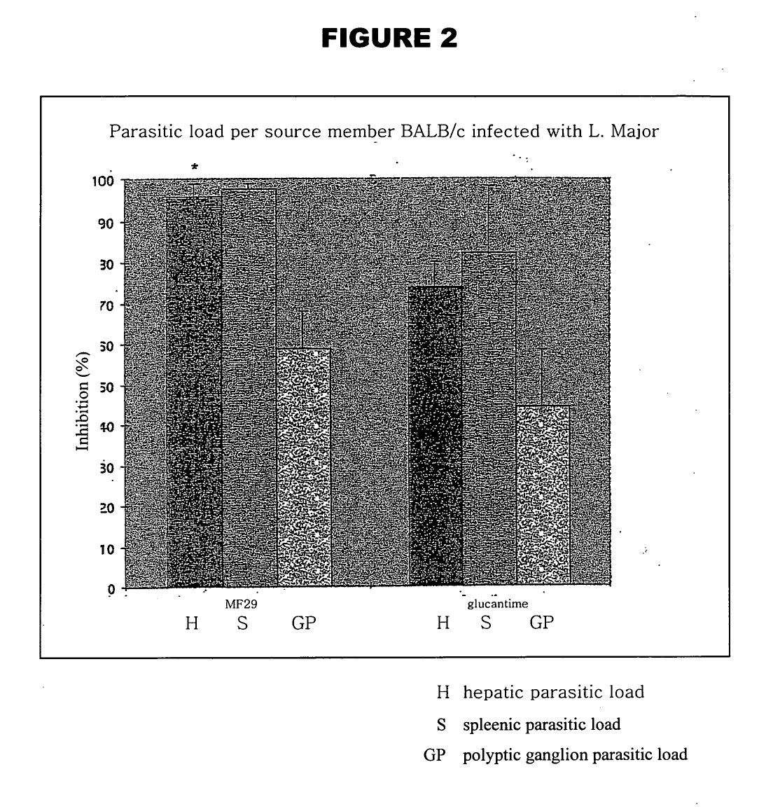

FIG. 2 represents the effects of MF 29 and Glucantime™ expressed in percentage of inhibition of the parasitic load per source member BALB/c infected with L. Major;

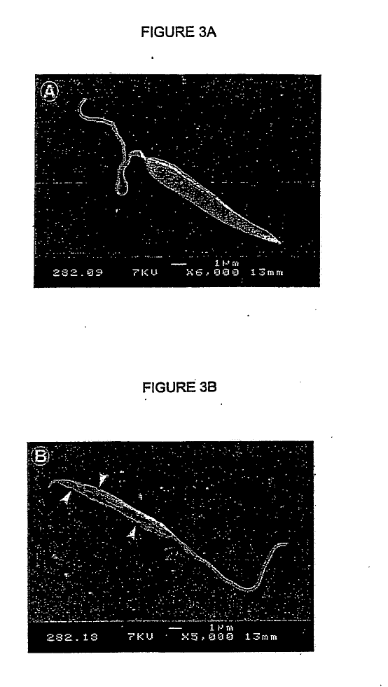

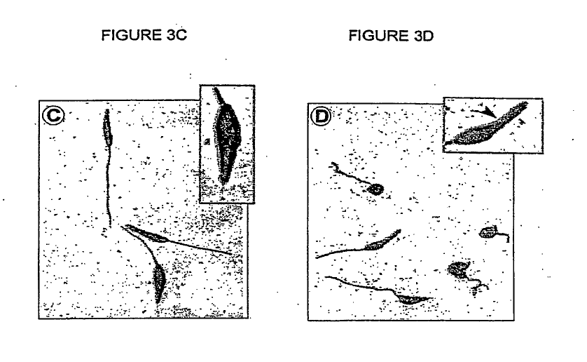

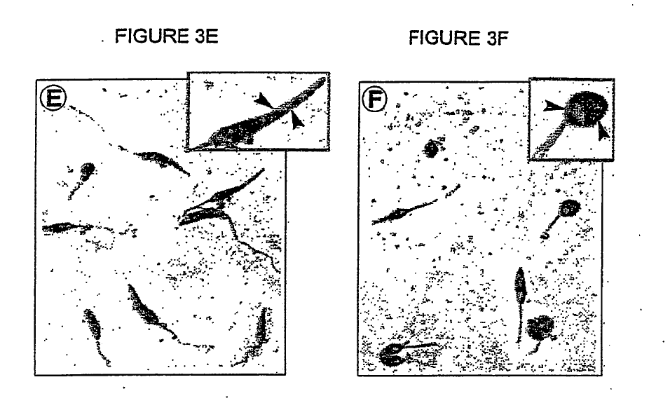

FIGS. 3A to 3F represent, in photographic form, the morphological changes induced by MF (FIGS. 3B, 3D, 3F) by comparison to a control group (FIGS. 3A, 3C, 3E);

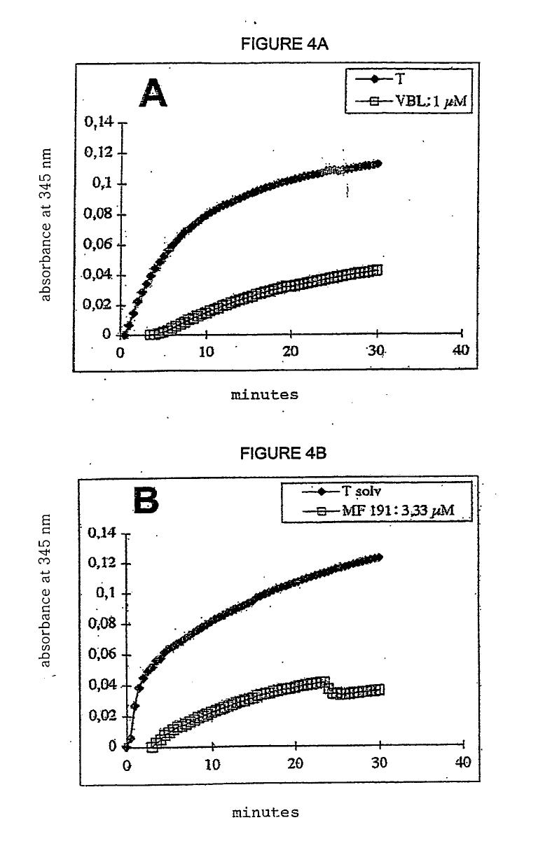

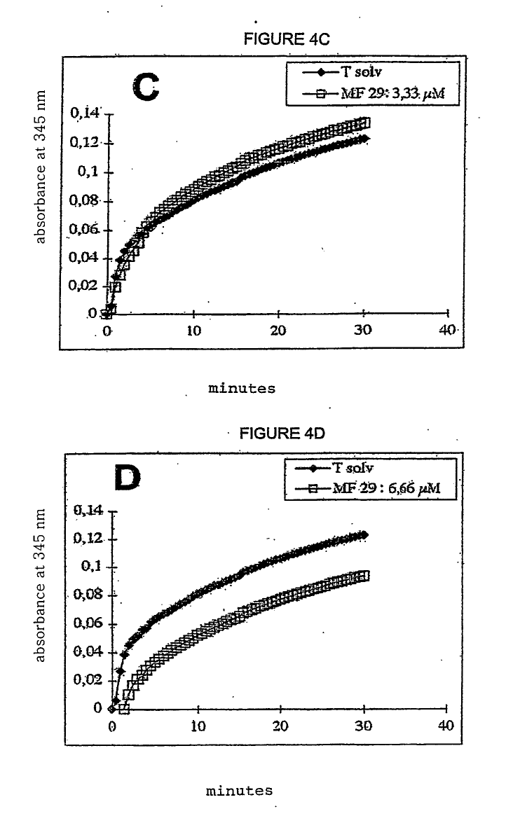

FIGS. 4A to 4D represent, in the form of a curve showing on the abscissa time expressed in minutes and on the ordinate absorbents at 345 nm, the effects of inhibition of the polymerization of tubuline of beef brain by vinblastine (FIG. 4A), MF 191 (FIG. 4B) and MF 29 at different dosages (FIG. 4C and 4D) and

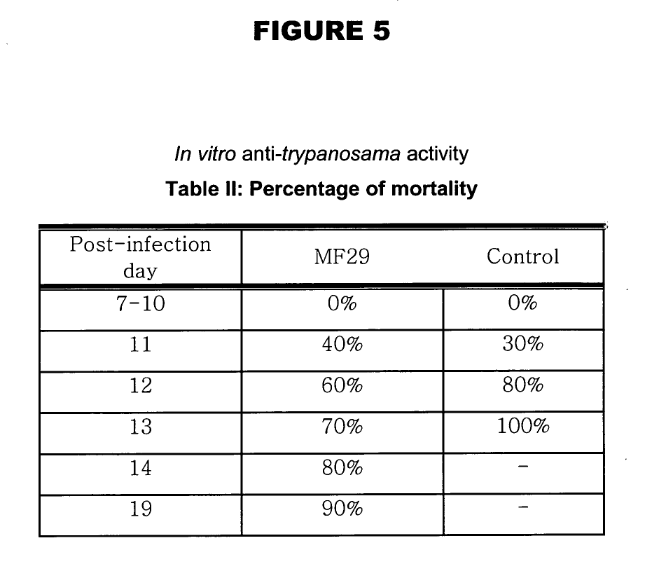

FIG. 5 represents, in tabular form, the percentages of mortality obtained on mice treated with MF 29 and infected by Trypanosoma cruzi.

By way of example and illustration of the invention, there are more particularly presented hereafter results obtained from molecules of the general formula (III), (IV), (V), (VI)

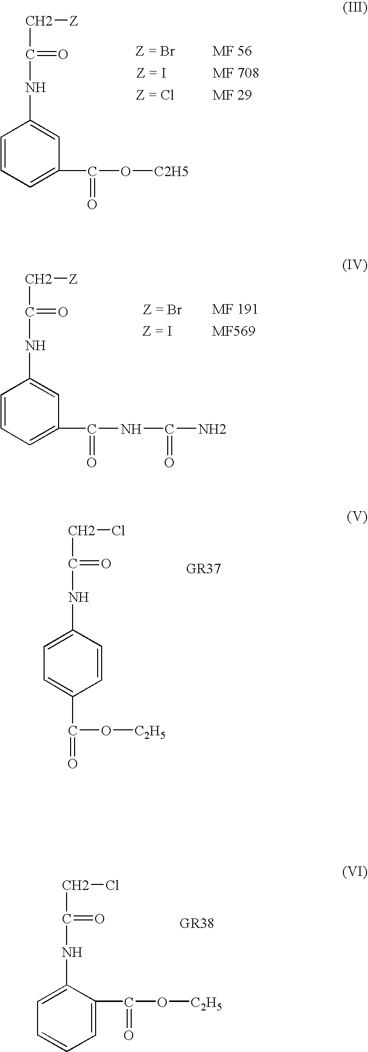

Thus:
the compound MF 191 corresponds to 3-bromoacetamido benzoyl urea,
MF 569 corresponds to 3-iodoacetamido benzoyl urea,
MF 708 corresponds to 3-iodoacetamido ethyl benzoate.
MF 56 corresponds to bromoacetamido ethyl benzoate,
MF 29 corresponds to 3-chloroacetamido ethyl benzoate,
GR 37 corresponds to 4-chloroacetamido ethyl benzoate and
GR 38 corresponds to 2-chloroacetamido ethyl benzoate.

There is described hereafter the in vitro anti-Leishmania activity of the molecules described above.

The above molecules have been evaluated first in a test of inhibition of proliferation of the promastigote phase of Leishmania after 96 hours of contact with promastigotes with molecules placed in solution in a solvent: N,N dimethyl acetamide/propylene glycol/Tween 80 (1:2:1). A tetrazolium salt (MTT) has been used as marker. In the case of L. mexicana, the molecules were evaluated relative to the intracellular amastigote stage (macrophages of Balb/c), the stage encountered in patients. Table 1 (FIG. 1) shows certain of the CI₅₀ of the 3-haloacetamido benzoate derivative, 3-haloacetamido ethyl benzoate derivative, 4-haloacetamido ethyl benzoate derivative and 2-chloroacetamido ethyl benzoate derivative as well as, for L. mexicana, those of the therapeutic reference molecule, meglumine antimoniate (GLUCANTIME)™. It is to be noted that the CI₅₀ expressed in μg of active molcule/ml of the solvent corresponds to the concentration of the compound inhibiting 50% of the proliferation of the leishmania parasite. These results show the high activity of MF 29, similar to that of GR 38, which is in the amastigote form 300 times greater than that of GLUCANTIME. There must however be noted the extraordinary activity of the compounds of the para form or 4-haloacetamido ethyl benzoate, as shown by GR 37 foretelling a high potential in the field of therapy of parasitic maladies. Finally, a test of cytotoxicity carried out on the MRC5 line (normal tissue of fetal lung) indicates that the cytotoxicity of MF 20 is 400 times less for this line than that obtained on the parasite, thereby demonstrating the low toxicity of MF 29 for human cells. Similarly, the cytotoxicity of GR37 is 1000 times less on mouse microphages than on the parasite, confirming the high potential of this molecule.

The in vivo anti-Leishmania activity of certain molecules, in particular that of MF 29, has also been examined.

In promastigotes of L. major (2×106) or of L. infantum (10⁷) in the stationary phase are inoculated respectively by the subcutaneous route in the rear right paw or by intravenous route in the caudal vein. The mice are treated by intra-peritoneal route once per day in the amount of 10 mg/kg of MF 29 in solution in a solvent: N,N dimethyl acetamide/propylene glycol/Tween 80 (1:2:1) for 10 days. Meglumine antimoniate (Glucantime-Speca™ Rhone-Poulenc, Paris, France) containing 85 mg of pentavalent antimony per ml is administered by subcutaneous route according to the same therapeutic scheme. The control group receives the solvent of MF 29 and of the saline solution at 0.9% by intra-peritoneal route.

For the cutaneous model, the efficacy of the treatment is evaluated by measuring the edema at the infected paw with the help of an electronic sliding foot (ref. 56371; Polylabo; Strasburg, France), and reported relative to the thickness of the counter-lateral paw. The mice are sacrificed nine months after infection. A fragment of derma, of the spleen, of the liver and of the polyptic ganglion draining the lesion are set aside to determine the parasitic load in situ. As to the model of visceral leishmaniosis, the same protocol is carried out on the spleen and the liver. The parasitic load is expressed by the log₁₀ of the number of parasites per gram of tissue.

These tests show high activity of the MF 29 compound on the infection with L. major in mice Balb/c. The treatment causes a reduction of the hepatic parasitic mode by 96%, of the spleen mode by 97% and of the polyptic ganglion mode by 59%. In this same model, the reference molecule, glucantime, causes a lesser reduction: the values are respectively 74.82 and 44% (FIG. 2). In the visceral model, glucantime and MF 29 have a similar efficiency. The reduction of the parasitic load in the liver is 25 and 30% whilst in the spleen, this reduction rises respectively to 56 and 66% for MF 29 and Glucantime.

In vivo studies, taking as the model the infected mice, confirm both the large cytotoxic effect on the proliferation of the parasite and the low toxicity of the product for mice.

The study of the morphological changes induced by MF 29 has also been undertaken.

After treatment for one hour with MF29 at 5 μg/ml and observation by sweeping electron microscope (JEOL, ISM 6400F), the promastigotes of L. mexicana have multiple pores (see the arrows) in the cellular body whilst, contrary to the other anti-tubuline drugs, the flagella do not seem to be disturbed [FIG. 3, control (A) and treated (B)].

Finally, the anti-microtubular activity of MF 29 has also been studied. There has also been studied in the first instance the alteration of the microtubular networks of promastigote of Leishmania after treatment with MF 29.

Antibodies directed against tubuline α and a protein immunologically connected to the neuronal protein MAP₂, have shown that (FIG. 3), under the influence of the drug, certain classes of microtubules were disorganized. The crown of sub-cortical microtubules was discontinuous [FIG. 3, control (C) and treated (D2)] and certain microtubules with the protein MAP₂ were partially depolymerized [FIG. 3, control (E) and treated (F)].

The technique used consisted in fixing the parasites, then incubating them in the presence of a first antibody (anti-tubuline a or anti-MAP₂) overnight, then the next day, after washing, incubating in the presence of a second antibody directed against the first, coupled to biotin. A streptavidine-peroxydase amplifying system is used, then the signal is detected by aminoethylcarbazole. The visualization of the parasitic cells is then carried out with a microscope connected to a tri-CDD camera (Lhesa electronic system, Rungis, France).

The anti-microtubular activity of MF 29 is also compared to that of MF 191 and to that of the vinblastine on the assembly of the tubulines of beef brain.

Experiments carried out to measure the capacity of in vitro polymerization of the tubuline were carried out with the help of a spectrophotometer provided with a circulating bath system at 37° C. and an automatic well loader permitting simultaneous measurement of several tests. The optical density which increases with polymerization of the assembly of the tubulines of the brain according to Lebanski et al., 1973, that is induced with the promoter MAP₂ is measured at 345 nm every thirty seconds. FIG. 4 shows that MF 29 is a weak inhibitor of polymerization of tubuline of beef brain (FIG. 4C). To have a significant inhibition (FIG. 4D), it is necessary to double the concentration of the drug. Even in this case, this inhibition remains less than that produced by MF 191 (FIG. 4B) or by vinblastine (FIG. 4A). MF 29 thus has a selective inhibitory action of the polymerization of tubuline by acting principally on the tubuline of the parasite and not on that of the host cell.

MF 29 thus has the advantage of not altering the microtubules of mammals. On the contrary, other anti-microtubularies with anti-parasitic activity are associated with high toxicity on human macrophages (tubulozoles) or on different lines of mammal cells (taxoides, zinc alkaloids). Moreover, the low levels of activity (high C150) of these compounds on the amastigote forms of Leishmania or on the parasitic tubuline indicate the high interest of the compounds described above for example.

Deep studies show that the mechanism of action of MF 29 involves a difference of activity according to the class of microtubules. Whilst the relation between heterogeneity of the microtubules and the action of the anti-microtubular drugs, such as tubulozoles, has never been studied, MF 29 has a more marked effect than the proteins associated with the tubulines (MAP) are bound to the microtubules. In this connection, there have been identified for the first time in Leishmania proteins immunologically related to MAP2. Knowing the important role of MAP2 in the processes of cellular division and differentiation, MF29 could act very effectively against these processes by altering the MAP2-like microtubules of the parasite.

The in vivo anti-Trypanosoma activity of MF 29 has also been studied. Mice were infected with Trypanosoma cruzi Peruvian strain by intravenous route. Four days post-infection, the mice are treated with MF 29 by intra-peritoneal route once per day in the amount of 10 mg/Kg for 10 days. The control group receives the solvent. The effectiveness of the treatment is evaluated by measuring parasitemia and followed by mortality. These tests show a maximum reduction of parasitemia at the 8^{th} day. Thus, the load is 200 trypomastigotes per 50 fields for the group treated with MF 29, as opposed to 680 in the control group, namely a reduction of the parasitic load of 70%. At the 10^{th} day, the parasitemia is equivalent for the two groups. The treatment moreover causes a retardation in the curve of mortality as shown in FIG. 5. The preliminary results of the use of MF 29 at 20 mg/kg show a greater reduction of mortality. Given the low doses used, satisfactory results could be obtained in the case of less virulent strains or for the above strains at higher doses.

The in vitro anti-Plasmodium activity of MF 29 has also been studied. MF 29 has been evaluated in a test of inhibition of the proliferation of Plasmodium falciparum (mortal species) in an intra-erythrocytic model by measuring the incorporation of hypoxanthine. MF 29 shows high activity with CI50 of 0.1 μg/ml on a chloroquinine-sensitive strain (FCB1-Columbia) but also on a strain (Nigerian strain) resistant to chloroquinine.

Several examples of pharmaceutical compositions are given hereafter:

### EXAMPLE A

Tablet for the treatment of parasitoses Compounds administered at 10 mg of active molecule

Preparation formula for 1000 tablets

| | |
|---|---|
| Active molecule | 10 g |
| Wheat starch | 35 g |
| Corn starch | 65 g |
| Lactose | 65 g |
| Magnesium stearate | 2 g |
| Silica | 1 g |
| Hydroxypropylcellulose | 2 g |

### EXAMPLE B

Ointment for the treatment of parasitoses

Formula for the preparation of 100 kg

Active molecule . . . 1000 g

Excipient in a quantity suffcient to make up 100 kg

The excipient can be selected from the following group:

Cetyl alcohol, sterile alcohol, isopropyl alcohol; lanolin, polyethylene glycol monostearate, distillate of cherry laurel.

It is quite evident that the above examples are given solely by way of illustration of the invention and not to limit this latter.

The present invention also has for its object a process for the inhibition of the polymerization of the tubuline by placing in contact with a system such as a parasite containing tubuline and inducing a parasitic malady with an effective quantity of a compound of the general formula I or II having the above structure.

The present invention also has for its object a process for the inhibition of the proliferation of parasites inducing leishmanioses, paludism and tripanosomiases, comprising the administration of a therapeutically active quantity of the anti-microtubular compounds of the general formula I or II described above.

The present invention also has for its object a process for the in vitro or in vivo inhibition of the growth and/or proliferation of parasites inducing parasitic maladies such as leishmanioses, paludism and tripanosomiases, this process comprising placing the parasites in contact with an effective quantity of an anti-

1-12. (canceled)

13. A method for administering an effective amount of a compound of formula (I): 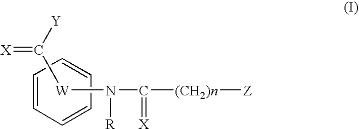
in which:
X represents oxygen, sulfur or nitrogen,
R represents hydrogen, a lower alkyl group,
n equals 0, 1, 2, 3, 4 or 5,
Z represents hydrogen, halogen, lower trifluoroalkyl, trifluoromethoxyl, cyano, hydroxyl, alkoxyl, alkoxycarbonyl, carboxamido, phenyl, substituted phenyl, halophenyl,
Y represents hydrogen, hydroxyl, alkoxyl, alkoxycarbonyl, carboxamido, phenyl, substituted phenyl, phenylalkyl, halophenyl, urea or a urea derivative, —N—R₁ in which R₁ is an alkyl group or a heterocyclic group,
the symbol 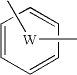
represents a method for treating parasites in a host cell by inhibiting the polymerization of tubulin of said parasite without inhibiting that of said host cell, comprising
either the phenyl nucleus
or the pyridine nucleus, the nitrogen being located at one of the 4 non-substituted positions,
its enantiomers, its diastereoisomers and its pharmaceutically acceptable acid addition salts for the production of an anti-microtubular agent active for the treatment of parasitic maladies by inhibiting at least partially the polymerization of the tubuline of the parasite without inhibiting that of the host cell.

14. The method according to claim 13, wherein said parasite is selected from the group consisting of protozooses, leishmaniosis, paludism and tripanosomiases.

15. A method for treating parasites in a patient comprising administering to said patient an effective amount of a compound of a formula (II) 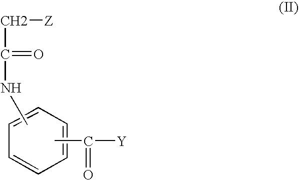
in which:
Z represents halogen selected from the group consisting of chlorine, bromine, iodine and fluorine,
Y is a substituent selected from the group consisting of ethoxy, —NH—CO—NH—R in which R is hydrogen, alkyl or aryl, and —N—R₁ in which R₁ is alkyl or heterocyclic,
or its addition salts.

16. The method according to claim 13, wherein
the compound is associated with at least one pharmaceutically acceptable non-toxic inert vehicle or excipient permitting its oral administration.

17. The method according to claim 16, in which the
compound is present in the form of a solution or an aqueous suspension or as dry tablets coated or not, gelatin coated tablets, capsules, powders.

18. The method according to claim 13,
in which the compound is associated with at least one pharmaceutically acceptable non-toxic inert excipient or vehicle permitting its administration topically in the form of a cutaneous, transcutaneous or percutaneous application.

19. The method according to claim 18,
in which the compound is in the form of an ointment, a cream or a dermal gel.

20. The method according to claim 15,
in which the compound is associated with at least one pharmaceutically acceptable non-toxic inert excipient or vehicle permitting its administration by parenteral, nasal or bronchial route.

21. The method according to claim 13,
in which the content of the compound is selected for administration ranging between 1 mg and 5 g/24 hours.

22. A compound of formula (I): 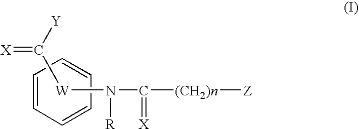
in which:
X represents oxygen, sulfur or nitrogen,
R represents hydrogen, lower alkyl,
n equals 0, 1, 2, 3, 4 or 5,
Z represents hydrogen, halogen, lower trifluoroalkyl, trifluoromethoxyl, cyano, hydroxyl, alkoxyl, alkoxycarbonyl, carboxamido, phenyl, substituted phenyl, halophenyl,
Y represents hydrogen, hydroxyl, alkoxyl, alkoxycarbonyl, carboxamido, phenyl, substituted phenyl, phenylalkyl, halophenyl, urea or a urea derivative, —N—R₁ in which R₁ is an alkyl or heterocyclic group,
the symbol 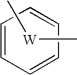
represents
either the phenyl nucleus
or the pyridine nucleus, the nitrogen being located at one of the 4 unsubstituted positions,
its enantiomers, its diastereoisomers, and its pharmaceutically acceptable acid addition salts
with the exception of compounds in which R is hydrogen, X is oxygen, n =1, Z is halogen, Y is a substituent selected from the group consisting of ethoxy, —NH—CO—NH—R in which R is hydrogen, alkyl or aryl, and by —N—R₁ in which R₁ is alkyl or heterocyclic or an addition salt of the latter.

23. A medicament, comprising,
a pharmaceutically acceptable support and a compound of formula (I) according to claim 22 or an addition salt of the latter in a quantity effective to treat parasitic maladies.
